(19) United States
(12) Patent Application Publication
(10) Pub. No.: US 2009/0053176 A1
(43) Pub. Date: Feb. 26, 2009

(54) New Combination 937

(75) Inventors: Gabrielle Ahlberg, Sodertalje (SE); Hakan Eriksson, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 12/195,505

(22) Filed: Aug. 21, 2008

Related U.S. Application Data
(60) Provisional application No. 60/957,524, filed on Aug. 23, 2007.

(51) Int. Cl. A61K 38/21

(52) U.S. Cl. 424/85.6

(57) ABSTRACT

The present invention related to a combination of (a) a compound which is a MPO inhibitor or a pharmaceutically acceptable salt thereof and (b) a compound or a pharmaceutically acceptable salt thereof, which is used in the treatment and/or prevention of PD or Multiple Sclerosis. The invention further relates to pharmaceutical compositions comprising said combination and to methods of treating Neuroinflammatory and Neurodegenerative Disorder(s), such as PD and Multiple Sclerosis in mammals by administrating said combination. The invention further relates to a kit comprising the combination and use of said kit in treatment of Neuroinflammatory Disorder(s).

### TECHNICAL FIELD

The present invention relates to combinations of (a) a first therapeutic agent which is a compound or a pharmaceutically acceptable salt thereof acting as inhibitor of the enzyme myeloperoxidase (MPO) and (b) a second therapeutic agent, which is a compound or a pharmaceutically acceptable salt thereof, which is known to be used in the treatment or prevention of Parkinson's disease (PD) or Multiple Sclerosis (MS). The present invention further provides pharmaceutical compositions comprising said combinations and to methods of treating neurological diseases, such as Parkinson's disease and Multiple Sclerosis, in mammals by administering said combinations. The present invention further relates to kits comprising said combinations and to the use of said kits in the treatment of neurological diseases, such as Parkinson's disease and multiple sclerosis.

### BACKGROUND OF THE INVENTION

Myeloperoxidase (MPO) is a heme-containing enzyme found predominantly in polymorphonuclear leukocytes (PMNs). MPO is one member of a diverse protein family of mammalian peroxidases that also includes eosinophil peroxidase, thyroid peroxidase, salivary peroxidase, lactoperoxidase, prostaglandin H synthase, and others. The mature enzyme is a dimer of identical halves. Each half molecule contains a covalently bound heme that exhibits unusual spectral properties responsible for the characteristic green colour of MPO. Cleavage of the disulphide bridge linking the two halves of MPO yields the hemi-enzyme that exhibits spectral and catalytic properties indistinguishable from those of the intact enzyme. The enzyme uses hydrogen peroxide to oxidize chloride to hypochlorous acid. Other halides and pseudohalides (like thiocyanate) are also physiological substrates to MPO.

PMNs are of particular importance for combating infections. These cells contain MPO, with well-documented microbicidal action. PMNs act non-specifically by phagocytosis to engulf microorganisms, incorporate them into vacuoles, termed phagosomes, which fuse with granules containing myeloperoxidase to form phagolysosomes. In phagolysosomes the enzymatic activity of the myeloperoxidase leads to the formation of hypochlorous acid, a potent bactericidal compound. Hypochlorous acid is oxidizing in itself, and reacts most avidly with thiols and thioethers, but also converts amines into chloramines, and chlorinates aromatic amino acids. Macrophages are large phagocytic cells, which, like PMNs, are capable of phagocytosing microorganisms. Macrophages can generate hydrogen peroxide and upon activation also produce myeloperoxidase. MPO and hydrogen peroxide can also be released to the outside of the cells where the reaction with chloride can induce damage to adjacent tissue.

Linkage of myeloperoxidase activity to disease has been implicated in neurological diseases with a neuroinflammatory response including multiple sclerosis, Alzheimer's disease and Parkinson's disease.

MPO positive cells are immensely present in the circulation and in tissue undergoing inflammation. More specifically MPO containing macrophages,microglia, astrocytes and/or neuronshave been documented in the CNS during disease; multiple sclerosis (Nagra R M, et al. Journal of Neuroimmunology 1997; 78(1-2):97-107; Marik C, et al. Brain 2007; 130: 2800-15; Gray E, et al. Brain Pathol 2008; 18: 86-95 ), Parkinson's disease (Choi D-K. et al. J. Neurosci. 2005; 25(28):6594-600) and Alzheimer's disease (Green P S. et al. Journal of Neurochemistry. 2004; 90(3):724-33). It is supposed that some aspects of a chronic ongoing inflammation result in an overwhelming destruction where agents from MPO reactions have an important role.

The enzyme is released both extracellularly as well as into phagolysosomes in the neutrophils (Hampton M B, Kettle A J, Winterbourn C C. Blood 1998; 92(9): 3007-17). The subcellular localization has been less well documented in other cell types but in human macrophages the MPO can be released extracellularly upon stimulation (Sugiyama S, et al. Am J Pathol 2001; 158: 879-91). A prerequisite for the MPO activity is the presence of hydrogen peroxide, generated by NADPH oxidase and a subsequent superoxide dismutation. The oxidized enzyme is capable to use a plethora of different substrates of which chloride is most recognized. From this reaction the strong non-radical oxidant—hypochlorous acid (HOCl)—is formed. HOCl oxidizes sulphur containing amino acids like cysteine and methionine very efficiently (Peskin A V, Winterbourn C C. Free Radical Biology and Medicine 2001; 30(5):572-9). It also forms chloramines with amino groups, both in proteins and other biomolecules (Peskin A V. et al. Free Radical Biology and Medicine 2004; 37(10):1622-30). It chlorinates phenols (like tyrosine) (Hazen S L. et al. Mass Free Radical Biology and Medicine 1997; 23(6):909-16) and unsaturated bonds in lipids (Albert C J. et al. J. Biol. Chem. 2001; 276(26):23733-41), oxidizes iron centers (Rosen H, Klebanoff S J. Journal of Biological Chemistry 1982; 257(22):13731-354) and crosslinks proteins (Fu X, Mueller D M, Heinecke J W. Biochemistry 2002; 41(4):1293-301).

Proteolytic cascades participate both in cell infiltration through the BBB (blood-brain-barrier) as well as the destruction of BBB, myelin and nerve cells (Cuzner M L, Opdenakker G. Journal of Neuroimmunology 1999; 94(1-2):1-14; Yong V W. et al. Nature Reviews Neuroscience 2001; 2(7):502-11.). Activation of matrix metalloproteinases (MMPs) can be accomplished through the action of upstream proteases in a cascade as well as through oxidation of a disulfide bridge Fu X. et al. J. Biol. Chem. 2001; 276(44):41279-87; Gu Z. et al. Science 2002; 297(5584):1186-90). This oxidation can be either a nitrosylation or HOCl-mediated oxidation. Both reactions can be a consequence of MPO activity. Several reports have suggested a role for MMP's in general and MMP-9 in particular as influencing cell infiltration as well as tissue damage (BBB breakdown and demyelination), both in MS and EAE (for review see Yong V W. et al, supra). The importance of these specific kinds of mechanisms in MS comes from studies where increased activity and presence of proteases have been identified in MS brain tissue and CSF. Supportive data has also been generated by doing EAE studies with mice deficient in some of the proteases implicated to participate in the MS pathology, or by using pharmacological approaches.

The demyelination is supposed to be dependent on the cytotoxic T-cells and toxic products generated by activated phagocytes (Lassmann H. J Neurol Neurosurg Psychiatry 2003; 74(6):695-7). The axonal loss is thus influenced by proteases and reactive oxygen and nitrogen intermediates. When MPO is present it will obviously have the capability of both activating proteases (directly as well as through disinhibition by influencing protease inhibitors) and generating reactive species.

Various compounds that are MPO inhibitors are disclosed in WO 01/85146, J. Heterocyclic Chemistry, 1992, 29, 343-354, J. Chem. Soc., 1962, 1863, WO03/089430 and WO2006/062465.

There is a constant need for new medications for the treatment or prevention of neurological diseases such as Parkinson's disease and multiple sclerosis. The combinations described herein are contemplated to provide synergistic or additive effects in treating said diseases. The method of treatment described herein will improve the effect of a compound or a pharmaceutically acceptable salt thereof, which is used in the treatment and/or prevention of Parkinson's disease or Multiple Sclerosis when taken in combination with a MPO inhibitor or a pharmaceutically acceptable salt thereof and, therefore permit improved management of disease progression and of symptoms and disease-related side effects. Other features and advantages will be apparent from the following detailed description and from the claims. A further advantage of this synergistic effect may be a quicker onset of the therapeutic effect of the single compounds.

### OUTLINE OF THE INVENTION

The present invention relates to a combination comprising (a) an amount of a first therapeutic agent, which is a MPO inhibitor or a pharmaceutically acceptable salt thereof and (b) an amount of a second therapeutic agent, which is a compound or a pharmaceutically acceptable salt thereof, which is used in the treatment and/or prevention of Parkinson's Disease or Multiple Sclerosis.

The present invention also relates to a pharmaceutical composition comprising a combination comprising (a) an amount of a first therapeutic agent, which is MPO inhibitor or a pharmaceutically acceptable salt thereof and (b) an amount of a second therapeutic agent, which is a compound or a pharmaceutically acceptable salt thereof, which is used in the treatment and/or prevention of Parkinson's Disease or Multiple Sclerosis, together with a pharmaceutically-acceptable vehicle, carrier or diluent.

One aspect of the present invention relates to a pharmaceutical composition, wherein the second therapeutic agent is selected from: the group of dopamine agonists, such as those known under the generic name of bromocriptine, pergolide, ropinirole, pramipexole, cabergoline, apomorphine, pribedile and rotigotine, or from the group of MAO-B inhibitors, such as those known under the generic names of selegiline and rasagiline, or from the group of other dopaminergic compounds, such as those known under the generic names of Tolcapone, Entacapone and Budipine, or from the group of adamantane derivate, such as those known under the generic name of amantadine hydrochloride, or from the group of dopamine precursors, such as those known under the generic name of levodopa and its combination with decarboxylase inhibitors (carbidopa and benserazide) and with decarboxylase inhibitors and COMT-inhibitors (Entacapone).

Another aspect of the present invention relates to a pharmaceutical composition, wherein the second therapeutic agent is selected from: the group of Interferons, such as those known under the generic names of interferon beta-1a, interferon beta-1b, and combinations thereof, or from the group of selective immunosuppressive compounds, such as those known under the generic names of glatiramer acetate and Natalizumab.

The present invention also relates to a kit comprising a dosage unit of mixture of a first therapeutic agent, which is a MPO inhibitor or a pharmaceutically acceptable salt thereof and a dosage unit of a second therapeutic agent, which is a compound or a pharmaceutically acceptable salt thereof, which is used in the treatment and/or prevention of Parkinson's Disease or Multiple Sclerosis, optionally with instructions for use.

The present invention also relates to a method for Neuroinflammatory Disorder(s) in a subject in need thereof comprising administering simultaneously, sequentially or separately, to said subject (a) an amount of first therapeutic agent, which is a MPO inhibitor or a pharmaceutically acceptable salt thereof and (b) an amount of a second therapeutic agent, which is a compound or a pharmaceutically acceptable salt thereof, which is used in the treatment and/or prevention of Parkinson's Disease or Multiple Sclerosis, wherein the amounts of (a) and (b) are together synergistically effective in the treatment.

One aspect of the present invention relates to a method, wherein said disorder is Multiple Sclerosis or Parkinson's Disease.

One embodiment of the present invention relates to a method, wherein the second therapeutic agent is selected from: the group of dopamine agonists, such as those known under the generic name of bromocriptine, pergolide, ropinirole, pramipexole, cabergoline, apomorphine, pribedile and rotigotine, or from the group of MAO-B inhibitors, such as those known under the generic names of selegiline and rasagiline, or from the group of other dopaminergic compounds, such as those known under the generic names of Tolcapone, Entacapone and Budipine, or from the group of adamantane derivative, such as those known under the generic name of amantadine hydrochloride, or from the group of dopamine precursors , such as those known under the generic name of levodopa and its combination with decarboxylase inhibitors (carbidopa and benserazide) and with decarboxylase inhibitors and COMT-inhibitors (entacapone).

One embodiment of the present invention relates to a method, wherein the second therapeutic agent is selected from: the group of Interferons, such as those known under the generic names of interferon beta-l a, interferon beta-lb, and combinations thereof, or from the group of selective immunosuppressive compounds, such as those known under the generic names of glatiramer acetate and Natalizumab.

One embodiment of the present invention relates to a method using the kit disclosed above.

One embodiment of the present invention relates to a method, wherein (a) an amount of a first therapeutic agent, and (b) an amount of a second therapeutic agent are administered simultaneously, sequentially or separately, to the subject in a pharmaceutical composition additionally comprising a pharmaceutically-acceptable vehicle, carrier or diluent, by a method selected from the group consisting of oral, transmucosal, transdermal, nasal, pulmonary, buccal, parenteral rectal, and sublingual administration.

One embodiment of the present invention relates to a method, wherein (a) an amount of a first therapeutic agent, and (b) an amount of a second therapeutic agent are administered simultaneously, sequentially or separately, to the subject in a pharmaceutical composition additionally comprising a pharmaceutically-acceptable vehicle, carrier or diluent, by a method selected from the group consisting of intravenous, intramuscular, subcutaneous, and intradermal.

The present invention also relates to the use of a combination comprising (a) an amount of a first therapeutic agent, which is a MPO inhibitor or a pharmaceutically acceptable salt and (b) an amount of a second therapeutic agent, which is a compound or a pharmaceutically acceptable salt thereof, in the which is used in the treatment and/or prevention of Parkinson's Disease or Multiple Sclerosis for neuroprotection.

Therapeutic Agents

is According to the present invention the first therapeutic agent is a compound that act as inhibitor of the enzyme myeloperoxidase (MPO).

According to one aspect of the present invention said first therapeutic agent is selected from a compound of formula (I)

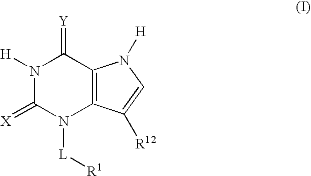

wherein:

at least one of X and Y represents S, and the other represents O or S;

L represents a direct bond or C₁₋₇alkylene, wherein said C₁₋₇alkylene optionally incorporating a heteroatom selected from O, S(O), and NR⁶, and said C₁₋₇alkylene optionally incorporating one or two carbon-carbon double bonds, and said C₁₋₇alkylene is optionally substituted by one or more substituents selected independently from OH, halogen, CN and NR⁴R⁵, C₁₋₆alkyl and C₁₋₆alkoxy, said C₁₋₆alkoxy optionally incorporating a carbonyl adjacent to the oxygen;

n represents an integer 0, 1 or 2;

R¹ is hydrogen, or

R¹ is a saturated or partially unsaturated 3 to 7 membered ring optionally incorporating one or two heteroatoms selected independently from O, N and S, and optionally incorporating a carbonyl group, wherein said ring is optionally substituted by one or more substituents independently selected from halogen, SO₂R⁹, SO₂NR⁹R¹⁰, OH, C₁₋₇alkyl, C₁₋₇alkoxy, CN, CONR²R³, NR²COR and COR³, wherein said C₁₋₇alkoxy being optionally further substituted by C₁₋₆alkoxy and optionally incorporating a carbonyl adjacent to the oxygen, and said C₁₋₇alkyl being optionally further substituted by hydroxy or C₁₋₆alkoxy and said C₁₋₇alkyl or C₁₋₆alkoxy optionally incorporating a carbonyl adjacent to the oxygen or at any position in the C₁₋₇alkyl; or

R¹ is an aromatic ring system selected from phenyl, biphenyl, naphthyl or a monocyclic or bicyclic heteroaromatic ring structure containing 1 to 3 heteroatoms independently selected from O, N and S, said aromatic ring system being optionally substituted by one or more substituents independently selected from halogen, SO₂R⁹, SO₂NR⁹R¹⁰, OH, C₁₋₇alkyl, C₁₋₇alkoxy, CN, CONR²R³, NR²COR³ and COR³; said C₁₋₇alkoxy being optionally further substituted by C₁₋₆alkoxy and said C₁₋₆alkoxy optionally incorporating a carbonyl adjacent to the oxygen, and said C₁₋₇alkyl being optionally further substituted by hydroxy or C₁₋₆alkoxy and said C₁₋₇alkyl or C₁₋₆alkoxy optionally incorporating a carbonyl adjacent to the oxygen or at any position in the alkyl;

R¹² represents hydrogen or halogen or a carbon optionally substituted with one to three halogen atoms;

at each occurrence, R², R³, R⁴, R⁵, R⁶, R⁹ and R¹⁰ independently represent hydrogen, C₁₋₆alkyl or C₁₋₆alkoxy said alkoxy optionally incorporating a carbonyl adjacent to the oxygen, said C₁₋₆alkyl being optionally further substituted by halogen, C₁₋₆alkoxy, CHO, C₂₋₆alkanoyl, OH, CONR⁷R⁸ and NR⁷COR⁸;

or the groups NR²R³, NR⁴R⁵ and NR⁹R¹⁰ each independently represent a 5 to 7 membered saturated azacyclic ring optionally incorporating one additional heteroatom selected from O, S and NR¹¹, said azacyclic ring being optionally further substituted by halogen, C₁₋₆alkoxy, CHO, C₂₋₆alkanoyl, OH, CONR⁷R⁸ and NR⁷COR⁸;

at each occurrence R⁷, R⁸ and R¹¹ independently represent hydrogen or C₁₋₆alkyl, or the group NR⁷R⁸ represents a 5- to 7-membered saturated azacyclic ring optionally incorporating one additional heteroatom selected from O, S and NR¹¹;

or pharmaceutically acceptable salts, solvates of solvates of salts thereof. These compounds are described in WO 2006/062465.

According to one aspect of the present invention said first therapeutic agent is selected from:

1-butyl-2-thioxo-1,2,3,5-tetrahydro-pyrrolo[3,2-d]pyrimidin-4-one;

1-isobutyl-2-thioxo-1,2,3,5-tetrahydro-pyrrolo[3,2-d]pyrimidin-4-one;

1-(pyridin-2-ylmethyl)-2-thioxo-1,2,3,5-tetrahydro-pyrrolo[3,2-d]pyrimidin-4-one;

1-(2-fluoro-benzyl)-2-thioxo-1,2,3,5-tetrahydro-pyrrolo[3,2-d]pyrimidin-4-one;

1-[2-(2-methoxyethoxy)-3-propoxybenzyl]-2-thioxo-1,2,3,5-tetrahydro-pyrrolo[3,2-d]pyrimidin-4-one;

1-(6-ethoxy-pyridin-2-ylmethyl)-2-thioxo-1,2,3,5-tetrahydro-pyrrolo[3,2-d]pyrimidin-4-one;

1-piperidin-3-ylmethyl-2-thioxo-1,2,3,5-tetrahydro-pyrrolo[3,2-d]pyrimidin-4-one;

1-butyl-4-thioxo-1,3,4,5-tetrahydro-2H-pyrrolo[3,2-d]pyrimidin-2-one;

1-(2-isopropoxyethyl)-2-thioxo-1,2,3,5-tetrahydro-pyrrolo[3,2-d]pyrimidin-4-one;

1-(2-methoxy-2-methylpropyl)-2-thioxo-1,2,3,5-tetrahydro-pyrrolo[3,2-d]pyrimidin-4-one;

1-(2-ethoxy-2-methylpropyl)-2-thioxo-1,2,3,5-tetrahydro-pyrrolo[3,2-d]pyrimidin-4-one;

1-(piperidin-4-ylmethyl)-2-thioxo-1,2,3,5-tetrahydro-pyrrolo[3,2-d]pyrimidin-4-one;

1-[(1-methylpiperidin-3-yl)methyl]-2-thioxo-1,2,3,5-tetrahydro-pyrrolo[3,2-d]pyrimidin-4-one;

1-[2-hydroxy-2-(4-methoxyphenyl)ethyl]-2-thioxo-1,2,3,5-tetrahydro-pyrrolo[3,2-d]pyrimidin-4-one;

1-(2-methoxybenzyl)-2-thioxo-1,2,3,5-tetrahydro-pyrrolo[3,2-d]pyrimidin-4-one;

1-(3-methoxybenzyl)-2-thioxo-1,2,3,5-tetrahydro-pyrrolo[3,2-d]pyrimidin-4-one;

1-(2,4-dimethoxybenzyl)-2-thioxo-1,2,3,5-tetrahydro-pyrrolo[3,2-d]pyrimidin-4-one;

1-[(3-chloropyridin-2-yl)methyl]-2-thioxo-1,2,3,5-tetrahydro-pyrrolo[3,2-d]pyrimidin-4-one;

1-{[3-(2-ethoxyethoxy)pyridin-2-yl]methyl}-2-thioxo-1,2,3,5-tetrahydro-pyrrolo[3,2-d]pyrimidin-4-one;

1-[(6-oxo-1,6-dihydropyridin-2-yl)methyl]-2-thioxo-1,2,3,5-tetrahydro-pyrrolo[3,2-d]pyrimidin-4-one;

1-(1H-indol-3-ylmethyl)-2-thioxo-1,2,3,5-tetrahydro-pyrrolo[3,2-d]pyrimidin-4-one;

1-(1H-benzimidazol-2-ylmethyl)-2-thioxo-1,2,3,5-tetrahydro-pyrrolo[3,2-d]pyrimidin-4-one;

1-[(5-chloro-1H-indol-2-yl)methyl]-2-thioxo-1,2,3,5-tetrahydro-pyrrolo[3,2-d]pyrimidin-4-one;

1-[(5-fluoro-1H-indol-2-yl)methyl]-2-thioxo-1,2,3,5-tetrahydro-pyrrolo[3,2-d]pyrimidin-4-one;

1-(1H-indol-6-ylmethyl)-2-thioxo-1,2,3,5-tetrahydro-pyrrolo[3,2-d]pyrimidin-4-one;

1-(1H-indol-5-ylmethyl)-2-thioxo-1,2,3,5-tetrahydro-pyrrolo[3,2-d]pyrimidin-4-one;

1-[(5-fluoro-1H-indol-3-yl)methyl]-2-thioxo-1,2,3,5-tetrahydro-pyrrolo[3,2-d]pyrimidin-4-one;

1-(1H-imidazol-5-ylmethyl)-2-thioxo-1,2,3,5-tetrahydro-pyrrolo[3,2-d]pyrimidin-4-one;

1-(1H-imidazol-2-ylmethyl)-2-thioxo-1,2,3,5-tetrahydro-pyrrolo[3,2-d]pyrimidin-4-one;

1-[(5-chloro-1H-benzimidazol-2-yl)methyl]-2-thioxo-1,2,3,5-tetrahydro-pyrrolo[3,2-d]pyrimidin-4-one;

1-[(4,5-dimethyl-1H-benzimidazol-2-yl)methyl]-2-thioxo-1,2,3,5-tetrahydro-pyrrolo[3,2-d]pyrimidin-4-one;

7-bromo-1-isobutyl-2-thioxo-1,2,3,5-tetrahydro-pyrrolo[3,2-d]pyrimidin-4-one; and

1-(3-chlorophenyl)-2-thioxo-1,2,3,5-tetrahydro-pyrrolo[3,2-d]pyrimidin-4-one;

or pharmaceutically acceptable salts thereof These compounds are described in WO 2006/062465.

In one aspect of the present invention said first therapeutic agent is selected from a compound of formula (IIa) or (IIb)

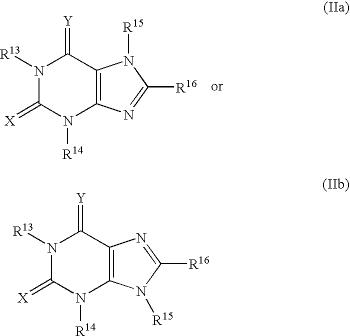

wherein:

one of X and Y represents S, and the other represents O or S;

R¹³ represents hydrogen or C₁₋₆alkyl;

R¹⁴ represents hydrogen or C₁₋₆alkyl; said C₁₋₆alkyl group being optionally substituted by:

i) a saturated or partially unsaturated 3- to 7-membered ring optionally incorporating one or two heteroatoms selected independently from O, N and S, and optionally incorporating a carbonyl group; said ring being optionally substituted by one or more substituents selected from halogen, hydroxy, C₁₋₆alkoxy and C₁₋₆alkyl; said C₁₋₆alkyl being optionally further substituted by hydroxy or C₁₋₆alkoxy; or

ii) C₁₆alkoxy; or

iii) an aromatic ring selected from phenyl, furyl or thienyl; said aromatic ring being optionally further substituted by halogen, C₁₋₆alkyl or C₁₋₆alkoxy;

R¹⁵ and R¹⁶ independently represent hydrogen or C₁₋₆alkyl;

or a pharmaceutically acceptable salt, solvate or solvate of a salt thereof. These compounds are described in WO 2003/089430.

According to one aspect of the present invention said first therapeutic agent is selected from a compound of formula (IIa) or (IIb)

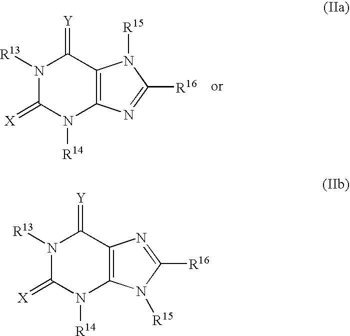

wherein:

X represents S, and Y represents O;

R¹³ represents hydrogen or C₁₋₆alkyl;

R¹⁴ represents C₁₋₆alkyl substituted by a saturated or partially unsaturated 3- to 7-membered ring optionally incorporating one or two heteroatoms selected independently from O, N and S, and optionally incorporating a carbonyl group; said ring being optionally substituted by one or more substituents selected from halogen, hydroxy, C₁₋₆alkoxy and C₁₋₆alkyl; said alkyl being optionally further substituted by hydroxy or C₁₋₆alkoxy;

R¹⁵ and R¹⁶ independently represent hydrogen or C₁₋₆alkyl;

or pharmaceutically acceptable salts, solvates or solvates of a salt thereof. These compounds are described in WO 2003/089430.

According to yet another aspect of the present invention, said first therapeutic agent is selected from:

1,3-diisobutyl-8-methyl-6-thioxanthine;

1,3-dibutyl-8-methyl-6-thioxanthine;

3-isobutyl-1,8-dimethyl-6-thioxanthine;

3-(2-methylbutyl)-6-thioxanthine;

3-isobutyl-8-methyl-6-thioxanthine;

3-isobutyl-2-thioxanthine;

3-isobutyl-2,6-dithioxanthine;

3-isobutyl-8-methyl-2-thioxanthine;

3-isobutyl-7-methyl-2-thioxanthine;

3-cyclohexylmethyl-2-thioxanthine;

3-(3-methoxypropyl)-2-thioxanthine;

3-cyclopropylmethyl-2-thioxanthine;

3-isobutyl-1-methyl-2-thioxanthine;

3-(2-tetrahydrofuryl-methyl)-2-thioxanthine;

3-(2-methoxy-ethyl)-2-thioxanthine;

3-(3-(1-morpholinyl)-propyl)-2-thioxanthine;

3-(2-furyl-methyl)-2-thioxanthine;

3-(4-methoxybenzyl)-2-thioxanthine;

3-(4-fluorobenzyl)-2-thioxanthine;

3-phenethyl-2-thioxanthine;

(+)-3-(2-tetrahydrofuryl-methyl)-2-thioxanthine;

(−)-3-(2-tetrahydrofuryl-methyl)-2-thioxanthine,

3-n-butyl-2-thioxanthine;

or a pharmaceutically acceptable salt, solvate or solvate of a salt thereof. These compounds are described in WO 2003/089430.

The (−)-enantiomer of 3-(2-tetrahydrofuryl-methyl)-2-thioxanthine represents 3-(2R-tetrahydrofuryl-methyl)-2-thioxanthine and the (+)-enantiomer of 3-(2-tetrahydrofuryl-methyl)-2-thioxanthine represents 3-(2S-tetrahydrofuryl-methyl)-2-thioxanthine.

According to one aspect of the present invention, the first therapeutic compound is a compound of Formula (III)

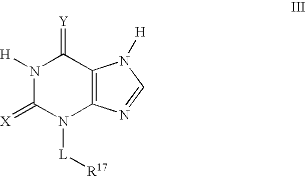

wherein

at least one of X and Y represents S, and the other represents O or S;

L represents (R¹⁸)ₚ-Q-(CR¹⁹R²⁰)ᵣ; wherein (R¹⁸)ₚ and (CR¹⁹R²⁰) each optionally contain one or two double or triple bonds;

wherein Q is O, S(O)ₙ, NR²¹, NR²¹C(O), C(O)NR²¹, or a bond;

wherein R¹⁸ is selected from C₁₋₆alkyl or C₁₋₆alkoxy, said C₁₋₆alkyl or said C₁₋₆alkoxy is optionally substituted with OH, halogen, CF₃, CHF₂, CFH₂, CN, NR²²R²³, phenoxy or aryl; and wherein said phenoxy is optionally substituted with C₁₋₆alkyl, halogen or C₁₋₆alkoxy; and wherein said phenoxy optionally incorporates a carbonyl adjacent to the oxygen and wherein said C₁₋₆alkoxy optionally incorporates a carbonyl adjacent to the oxygen;

wherein R¹⁹ and R²⁰ are independently selected from hydrogen, OH, halogen, CF₃, CHF₂, CFH₂, CN, NR²²R²³, C1 to 6 alkyl, phenoxy and C₁₋₆alkoxy; wherein said phenoxy or C₁₋₆alkoxy optionally incorporates a carbonyl adjacent to the oxygen; and wherein said phenoxy is optionally substituted with C₁₋₆alkyl, halogen or C₁₋₆alkoxy;

wherein p represents an integer 0, 1, 2, 3 or 4 and r represents an integer 0, 1, 2, 3 or 4; and

wherein 1≦p+r≦7;

R¹⁷ represents a mono- or bicyclic heteroaromatic ring system containing one or more heteroatoms selected from N, O and S; wherein said mono- or bicyclic heteroaromatic ring system is optionally fused with one or two 5- or 6-membered saturated or partially saturated ring(s) containing one or more atoms selected from C, N, O and S, wherein said mono- or bicyclic heteroaromatic ring system alone or when fused with one or two 5- or 6-membered saturated or partially saturated ring(s) is optionally substituted with one or more substituents independently selected from halogen, CHF₂, CH₂F, CF₃, SO₍ₙ₎R²⁴, SO₍ₙ₎NR²⁴R²⁵, (CH₂)R²⁶, NR²²R²³, OH, C1 to 7 alkyl, C₁₋₇alkoxy, phenoxy, aryl, CN C(O)NR²⁷R²⁶, NR²C(O)R²⁶, C(O)R²⁶, a 5- or 6-membered saturated or partially saturated ring containing one or more atoms selected from C, N, O or S, and a mono- or bicyclic heteroaromatic ring system containing one or more heteroatoms selected from N, S or O; and wherein said C₁₋₇alkoxy is optionally substituted with C₁₋₇alkoxy or aryl; and wherein said C₁₋₇alkoxy or said phenoxy is optionally incorporating a carbonyl adjacent to the oxygen; and wherein said C₁₋₇alkyl is optionally substituted with hydroxy or C₁₋₆alkoxy; and wherein said C₁₋₇alkyl is optionally incorporating a carbonyl at any position in the C C₁₋₇alkyl; and wherein said phenoxy is optionally substituted with C₁₋₆alkyl, halogen or C₁₋₆alkoxy;

at each occurrence, R²⁷, R²⁶, R²², R²³, R²¹, R²⁴ and R²⁵ are independently selected from hydrogen, C₁₋₆alkyl, C₁₋₆alkoxy, aryl and phenoxy; said C₁₋₆alkoxy or phenoxy is optionally incorporating a carbonyl adjacent to the oxygen; and said C₁₋₆alkyl is optionally substituted with halogen, C₁₋₆alkoxy, CHO, C₂₋₆alkanoyl, OH, C(O)NR²⁸R²⁹ or NR²⁸C(O)R²⁹; and said aryl or said phenoxy is optionally substituted with C₁₋₆alkyl, halogen or C₁₋₆alkoxy;

or the groups NR²⁷R²⁶, NR²²R²³ and NR²⁴R²⁵ each independently represents a 5 to 7 membered saturated azacyclic ring optionally incorporating one additional heteroatom selected from O, S and NR³⁰, said ring being optionally further substituted with halogen, C₁₋₆alkoxy, CHO, C₂₋₆alkanoyl, OH, C(O)NR²⁸R²⁹ or NR²⁸C(O)R²⁹;

at each occurrence R²⁸, R²⁹ and R³⁰ independently represent hydrogen or C₁₋₆alkyl, or the group NR²⁸R²⁹ represents a 5 to 7 membered saturated azacyclic ring optionally incorporating one additional heteroatom selected from O, S and NR³⁰;

n represents an integer 0, 1 or 2;

with the proviso that for R¹⁷ thienyl or furyl is excluded;

and with the proviso that when Q is O, S(O)ₙ, NR²¹, NR²¹C(O) or C(O)NR²¹, then p is greater or equal to 1;

or a pharmaceutically acceptable salt, solvate or solvate of a salt thereof. These compounds are described in PCT/SE2007/000349.

According to yet another aspect of the present invention, said first therapeutic compound is selected from:

3-(pyridin-2-ylmethyl)-2-thioxo-1,2,3,7-tetrahydro-6H-purin-6-one;

3-(pyridin-3-ylmethyl)-2-thioxo-1,2,3,7-tetrahydro-6H-purin-6-one;

3-(pyridin-4-ylmethyl)-2-thioxo-1,2,3,7-tetrahydro-6H-purin-6-one;

3-{[3-ethoxy-4-(2-ethoxyethoxy)pyridin-2-yl]methyl}-2-thioxo-1,2,3,7-tetrahydro-6H-purin-6-one;

3-[(5-fluoro-1H-indol-2-yl)methyl]-2-thioxo-1,2,3,7-tetrahydro-6H-purin-6-one;

3-[(5-fluoro-1H-indol-2-yl)methyl]-2-thioxo-1,2,3,7-tetrahydro-6H-purin-6-one;

3-[(2-butyl-4-chloro-1H-imidazol-5-yl)methyl]-2-thioxo-1,2,3,7-tetrahydro-6H-purin-6-one;

3-(1H-benzimidazol-2-ylmethyl)-2-thioxo-1,2,3,7-tetrahydro-6H-purin-6-one;

3-[1-(1H-benzimidazol-2-yl)ethyl]-2-thioxo-1,2,3,7-tetrahydro-6H-purin-6-one;

3-[(5-chloro-1H-indol-3-yl)methyl]-2-thioxo-1,2,3,7-tetrahydro-6H-purin-6-one and

3-[(4-fluoro-1H-indol-3-yl)methyl]-2-thioxo-1,2,3,7-tetrahydro-6H-purin-6-one;

3-[2-(1H-Benzimidazol-2-yl)ethyl]-2-thioxo-1,2,3,7-tetrahydro-6H-purin-6-one;

3-(1H-Pyrazol-3-ylmethyl)-2-thioxo-1,2,3,7-tetrahydro-6H-purin-6-one;

3-[(5-Methylpyrazin-2-yl)methyl)-2-thioxo-1,2,3,7-tetrahydro-6H-purin-6-one;

3-[(3-Isopropylisoxazol-5-yl)methyl]-2-thioxo-1,2,3,7-tetrahydro-6H-purin-6-one;

3-[(4-Methyl-1,2,5-oxadiazol-3-yl)methyl]-2-thioxo-1,2,3,7-tetrahydro-6H-purin-6-one;

3-[(6-Butoxypyridin-2-yl)methyl]-2-thioxo-1,2,3,7-tetrahydro-6H-purin-6-one;

3-[(4-Butoxypyridin-2-yl)methyl]-2-thioxo-1,2,3,7-tetrahydro-6H-purin-6-one;

3-[(3-Butoxypyridin-2-yl)methyl]-2-thioxo-1,2,3,7-tetrahydro-6H-purin-6-one;

3-[2-(Pyridin-2-ylmethoxy)propyl]-2-thioxo-1,2,3,7-tetrahydro-6H-purin-6-one;

3-[(3,5-Dimethylisoxazol-4-yl)methyl]-2-thioxo-1,2,3,7-tetrahydro-6H-purin-6-one;

3-[(1-Methyl-1H-indol-2-yl)methyl]-2-thioxo-1,2,3,7-tetrahydro-6H-purin-6-one;

3-(2-Phenyl-2-pyridin-2-ylethyl)-2-thioxo-1,2,3,7-tetrahydro-6H-purin-6-one;

3-(Quinolin-4-ylmethyl)-2-thioxo-1,2,3,7-tetrahydro-6H-purin-6-one;

3-[(6-Phenoxypyridin-3-yl)methyl]-2-thioxo-1,2,3,7-tetrahydro-6H-purin-6-one;

3-{2-[(Quinolin-4-ylmethyl)amino]ethyl}-2-thioxo-1,2,3,7-tetrahydro-6H-purin-6-one;

3-(2-{[(1-Methyl-1H-indol-3-yl)methyl]amino } ethyl)-2-thioxo-1,2,3,7-tetrahydro-6H-purin-6-one;

3-{2-[Methyl(quinolin-4-ylmethyl)amino]ethyl}-2-thioxo-1,2,3,7-tetrahydro-6H-purin-6-one;

3-(2-Aminopropyl)-2-thioxo-1,2,3,7-tetrahydro-6H-purin-6-one trifluoroacetate;

3-{2-[(Pyridin-2-ylmethyl)amino]propyl}-2-thioxo-1,2,3,7-tetrahydro-6H-purin-6-one trifluoroacetate;

3-{2-[(Pyridin-3-ylmethyl)amino]propyl}-2-thioxo-1,2,3,7-tetrahydro-6H-purin-6-one;

3-{2-[(Pyridin-4-ylmethyl)amino]propyl}-2-thioxo-1,2,3,7-tetrahydro-6H-purin-6-one;

3-(2-{[(6-Chloropyridin-3-yl)methyl]amino}propyl)-2-thioxo-1,2,3,7-tetrahydro-6H-purin-6-one trifluoroacetate;

3-[2-({[6-(Trifluoromethyl)pyridin-3-yl]methyl}amino)propyl]-2-thioxo-1,2,3,7-tetrahydro-6H-purin-6-one trifluoroacetate;

3-(2-{[(4,6-Dichloropyrimidin-5-yl)methyl]amino}propyl)-2-thioxo-1,2,3,7-tetrahydro-6H-purin-6-one;

3-[2-({[2-(Dimethylamino)pyrimidin-5-yl]methyl}amino)propyl]-2-thioxo-1,2,3,7-tetrahydro-6H-purin-6-one;

3-{2-[(Quinolin-2-ylmethyl)amino]propyl}-2-thioxo-1,2,3,7-tetrahydro-6H-purin-6-one trifluoroacetate;

3-{2-[(Quinolin-3-ylmethyl)amino]propyl}-2-thioxo-1,2,3,7-tetrahydro-6H-purin-6-one;

3-(2-{[(1-tert-Butyl-3,5-dimethyl-1H-pyrazol-4-yl)methyl]amino }propyl)-2-thioxo-1,2,3,7-tetrahydro-6H-purin-6-one;

3-[2-({[1-(1,1-Dioxidotetrahydro-3-thienyl)-3,5-dimethyl-1H-pyrazol-4-yl]methyl}amino)propyl]-2-thioxo-1,2,3,7-tetrahydro-6H-purin-6-one;

3-{2-[(1H-Benzoimidazol-2-ylmethyl)amino]propyl}-2-thioxo-1,2,3,7-tetrahydro-6H-purin-6-one;

3-[2-({[1-(Phenylsulfonyl)-1H-pyrrol-2-yl]methyl}amino]propyl]-2-thioxo-1,2,3,7-tetrahydro-6H-purin-6-one trifluoroacetate;

3-{2-[({1-[(4-methylphenyl)sulfonyl]-1H-pyrrol-2-yl}methyl)amino]propyl}-2-thioxo-1,2,3,7-tetrahydro-6H-purin-6-one trifluoroacetate;

3-(2-{[(1-methyl-1H-pyrrol-2-yl)methyl]amino}propyl)-2-thioxo-1,2,3,7-tetrahydro-6H-purin-6-one;

3-[2-({[1-(4-sec-Butylphenyl)-1H-pyrrol-2-yl]methyl}amino)propyl)-2-thioxo-1,2,3,7-tetrahydro-6H-purin-6-one;

3-[2-({[1-(3-Methoxyphenyl)-1H-pyrrol-2-yl]methyl}amino)propyl]-2-thioxo-1,2,3,7-tetrahydro-6H-purin-6-one;

3-[2-({[2,5-Dimethyl-1-(1,3-thiazol-2-yl)-1H-pyrrol-3-yl]methyl}amino)propyl]-2-thioxo-1,2,3,7-tetrahydro-6H-purin-6-one;

3-[2-({[4-(3-Chlorobenzoyl)-1-methyl-1H-pyrrol-2-yl]methyl}amino)propyl]-2-thioxo-1,2,3,7-tetrahydro-6H-purin-6-one;

3-{2-[(1H-Imidazol-2-ylmethyl)amino]propyl}-2-thioxo-1,2,3,7-tetrahydro-6H-purin-6-one;

3-(2-{[(1-Methyl-1H-imidazol-2-yl)methyl]amino}propyl)-2-thioxo-1,2,3,7-tetrahydro-6H-purin-6-one;

3-(2-{[(4-Bromo-1-methyl-1H-imidazol-5-yl)methyl]amino}propyl)-2-thioxo-1,2,3,7-tetrahydro-6H-purin-6-one;

3-(2-{[(1-Methyl-1H-indol-3-yl)methyl]amino}propyl)-2-thioxo-1,2,3,7-tetrahydro-6H-purin-6-one;

2-Thioxo-3-{2-[(1H-1,2,3-triazol-5-ylmethyl)amino]propyl}-1,2,3,7-tetrahydro-6H-purin-6-one;

3-[2-({[1-(Benzyloxy)-1H-imidazol-2-yl]methyl}amino)propyl]-2-thioxo-1,2,3,7-tetrahydro-6H-purin-6-one;

3-(2-{[(6-Bromo-2-methylimidazo[1,2-a]pyridin-3-yl)methyl]amino}propyl}-2-thioxo-1,2,3,7-tetrahydro-6H-purin-6-one;

3-{2-[({1-[2-(2-Methoxyphenoxy)ethyl]-1H-pyrrol-2-yl}methyl)amino]propyl]-2-thioxo-1,2,3,7-tetrahydro-6H-purin-6-one;

N-[1-Methyl-2-(6-oxo-2-thioxo-1,2,6,7-tetrahydro-3H-purin-3-yl)ethyl]pyridine-2-carboxamide;

N-[1-Methyl-2-(6-oxo-2-thioxo-1,2,6,7-tetrahydro-3H-purin-3-yl)ethyl]nicotinamide;

N-[1-Methyl-2-(6-oxo-2-thioxo-1,2,6,7-tetrahydro-3H-purin-3-yl)-ethyl]isonicotinamide;

N-[1-methyl-2-(6-oxo-2-thioxo-1,2,6,7-tetrahydro-3H-purin-3-yl)ethyl]-1,8-naphthyridine-2-carboxamide;

N-[1-Methyl-2-(6-oxo-2-thioxo-1,2,6,7-tetrahydro-3H-purin-3-yl)ethyl]quinoline-2-carboxamide;

N-[1-Methyl-2-(6-oxo-2-thioxo-1,2,6,7-tetrahydro-3H-purin-3-yl)ethyl]pyrimidine-2-carboxamide; and

N-[1-Methyl-2-(6-oxo-2-thioxo-1,2,6,7-tetrahydro-3H-purin-3-yl)ethyl]-1H-imidazole-2-carboxamide trifluroaceate;

or a pharmaceutically acceptable salt, solvate or solvate of a salt thereof. These compounds are described in PCT/SE2007/000349.

According to the present invention, the second therapeutic agent is selected either from compounds or pharmaceutically acceptable salts thereof which are know to be used in the treatment of Parkinson's disease or from compounds or pharmaceutically acceptable salts thereof which are know to be used in the treatment of Multiple sclerosis.

Compounds or pharmaceutically salts thereof, which are known in the treatment of Parkinson's disease, are selected from:
1. dopamine agonists, such as those known under the generic name of bromocriptine, pergolide, ropinirole, pramipexole, cabergoline, apomorphine, pribedile and rotigotine.
2. MAO-B inhibitors, such as those known under the generic names of selegiline and rasagiline.
3. other d dopaminergic compounds, such as those known under the generic names of Tolcapone, Entacapone and Budipine.
4. adamantane derivate, such as those known under the generic name of amantadine hydrochloride
5. dopamine precursors, such as those known under the generic name of levodopa and its combination with decarboxylase inhibitors (carbidopa and benserazide) and with decarboxylase inhibitors and COMT-inhibitors (Entacapone)

Compounds or pharmaceutically salts thereof, which are known in the treatment of multiple sclerosis, are selected from:
1. Interferons, such as those known under the generic names of, interferon beta-1a, interferon beta-1b and combinations thereof.
2. Selective immunosuppressive compounds, such as those known under the generic names of glatiramer acetate and Natalizumab.

Combination

A first aspect of the present invention relates to a combination comprising (a) an amount of a first therapeutic agent, which is a MPO inhibitor or a pharmaceutically acceptable salt thereof and (b) an amount of a second therapeutic agent, which is a compound or a pharmaceutically acceptable salt thereof, which is used in the treatment and/or prevention of PD or MS. In one embodiment of the present invention the combination comprises the first therapeutic agent comprises one of the compounds selected from the groups defined above and the second therapeutic agent comprises one of the compounds selected from the groups defined above.

Pharmaceutical Composition

A second aspect of the present invention relates to a pharmaceutical composition comprising a combination comprising (a) an amount of a first therapeutic agent, which is MPO inhibitor or a pharmaceutically acceptable salt thereof and (b) an amount of a second therapeutic agent, which is a compound or a pharmaceutically acceptable salt thereof, which is used in the treatment and/or prevention of PD or MS, together with a pharmaceutically-acceptable vehicle, carrier or diluent.

In one embodiment of the present invention the first therapeutic agent comprises one of the compounds, which are MPO inhibitors or a pharmaceutically acceptable salt thereof, which are defined above.

In one embodiment of the present invention the second therapeutic agent comprises of a compound or a pharmaceutically acceptable salt thereof, which are used for the treatment and/or prevention of PD or MS as defined above. According to one embodiment of the present invention, said second therapeutic agent is selected from the group of dopamine agonists, such as those known under the generic name of bromocriptine, pergolide, ropinirole, pramipexole, cabergoline, apomorphine, pribedile and rotigotine. According to one embodiment of the present invention, said second therapeutic agent is selected from the group of MAO-B inhibitors, such as those known under the generic names of selegiline and rasagiline. According to one embodiment of the present invention, said second therapeutic agent is selected from the group of other dopaminergic compounds, such as those known under the generic names of Tolcapone, Entacapone and Budipine. According to one embodiment of the present invention, said second therapeutic agent is selected from the group of adamantane derivate, such as those known under the generic name of amantadine hydrochloride. According to one embodiment of the present invention, said second therapeutic agent is selected from the group of dopamine precursors, such as those known under the generic name of levodopa and its combination with decarboxylase inhibitors (carbidopa and benserazide) and with decarboxylase inhibitors and COMT-inhibitors (Entacapone). According to one embodiment of the present invention, said second therapeutic agent is selected from the group of Interferons, such as those known under the generic names of interferon beta-1a, interferon beta-1b, and combinations thereof. According to one embodiment of the present invention, said second therapeutic agent is selected from the group of selective immunosuppressive compounds, such as those known under the generic names of glatiramer acetate and Natalizumab.

For use in medicine, pharmaceutically acceptable salts may be useful in the preparation of the compounds according to the present invention. Suitable pharmaceutically acceptable salts of the compounds described herein include acid addition salts which may, for example, be formed by mixing a solution of the compound according to the present invention with a solution of a pharmaceutically acceptable acid such as hydrochloric acid, sulfuric acid, methanesulphonic acid and fumaric acid. Furthermore, where the compounds carry an acidic moiety, suitable pharmaceutically acceptable salts thereof may include alkali metal salts, e.g. sodium or potassium salts; alkaline earth metal salts, e.g. calcium or magnesium salts; and salts formed with suitable organic ligands, e.g. quaternary ammonium salts.

The expression “pharmaceutically acceptable salts” includes both pharmaceutically acceptable acid addition salts and pharmaceutically acceptable cationic salts. The expression “pharmaceutically acceptable cationic salts” is intended to define but is not limited to such salts as the alkali metal salts, (e.g., sodium and potassium), alkaline earth metal salts (e.g., calcium and magnesium), aluminum salts, ammonium salts, and salts with organic amines such as benzathine (N,N′-dibenzylethylenediamine) and choline. The expression “pharmaceutically acceptable acid addition salts” is intended to define but is not limited to such salts as the hydrochloride, hydrobromide and sulfate.

The pharmaceutically acceptable cationic salts containing free carboxylic acids can be readily prepared by reacting the free acid form of with an appropriate base. Typical bases are sodium hydroxide, sodium methoxide and sodium ethoxide.

The pharmaceutically acceptable acid addition salts containing free amine groups can be readily prepared by reacting the free base form with the appropriate acid.

The active ingredients of the composition described herein can be co-administered simultaneously or may be administered separately or sequentially in any order, or as a single pharmaceutical composition comprising the two therapeutic agents mentioned above.

The combinations described herein can be administered in a standard manner for the treatment of PD or MS such as orally, parenterally, transmucosally (e.g., sublingually or via buccal administration), topically, transdermally, rectally, via inhalation (e.g., nasal or deep lung inhalation). Parenteral administration includes, but is not limited to intravenous, intraarterial, intraperitoneal, subcutaneous, intramuscular, intrathecal or via a high-pressure technique.

For buccal administration, the composition can be in the form of tablets or lozenges formulated in conventional manner. For example, tablets and capsules for oral administration can contain conventional excipients such as binding agents (e.g., syrup, acacia, gelatin, sorbitol, tragacanth, mucilage of starch or polyvinylpyrrolidone), fillers (e.g., lactose, sugar, microcrystalline cellulose, maize-starch, calcium phosphate or sorbitol), lubricants (e.g., magnesium stearate, stearic acid, talc, polyethylene glycol or silica), disintegrants (e.g., potato starch or sodium starch glycollate), or wetting agents (e.g., sodium lauryl sulfate). Tablets may be coated according to methods well known in the art.

Such preparations can also be formulated as suppositories for rectal administration, e.g., containing conventional suppository bases, such as cocoa butter or other glycerides. Compositions for inhalation typically can be provided in the form of a solution, suspension, or emulsion that can be administered as a dry powder or in the form of an aerosol using a conventional propellant, such as dichlorodifluoromethane or trichlorofluoromethane. Typical topical and transdermal formulations comprise conventional aqueous or non-aqueous vehicles, such as eye drops, creams, ointments, lotions, and pastes, or are in the form of a medicated plaster, patch, or membrane.

Additionally, compositions described herein can be formulated for parenteral administration by injection or continuous infusion. Formulations for injection can be in the form of suspensions, solutions, or emulsions in oily or aqueous vehicles, and can contain formulation agents, such as suspending, stabilizing, and/or dispersing agents. Alternatively, the active ingredient can be in powder form for constitution with a suitable vehicle (e.g., sterile, pyrogen-free water) before use.

A composition in accordance with the present invention also can be formulated as a depot preparation. Such long acting formulations can be administered by implantation (e.g., subcutaneously or intramuscularly) or by intramuscular injection. Accordingly, the compounds of the present invention can be formulated with suitable polymeric or hydrophobic materials (e.g., an emulsion in an acceptable oil), ion exchange resins, or as sparingly soluble derivatives (e.g., a sparingly soluble salt).

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc may be used to form tablets. Solid compositions of a similar type are also employed as fillers in soft and hard-filled gelatin capsules; preferred materials in this connection also include lactose or milk sugar as well as high molecular weight polyethylene glycols.

Alternatively, the composition described herein can be incorporated into oral liquid preparations such as aqueous or oily suspensions, solutions, emulsions, syrups, or elixirs, for example. Moreover, formulations containing these compounds can be presented as a dry product for constitution with water or other suitable vehicle before use. Such liquid preparations can contain conventional additives, such as suspending agents, such as sorbitol syrup, synthetic and natural gums such as tragacanth, acacia, alginate, dextran, sodium carboxymethylcellulose, methylcellulose, polyvinyl-pyrrolidone or gelatin, glucose/sugar syrup, gelatin, hydroxyethylcellulose, hydroxypropylmethylcellulose, aluminum stearate gel, emulsifying agents, such as lecithin, sorbitan monooleate, or acacia; non-aqueous vehicles (which can include edible oils), such as almond oil, fractionated coconut oil, oily esters, propylene glycol, and ethyl alcohol; and preservatives, such as methyl or propyl p-hydroxybenzoate and sorbic acid. The liquid forms in which the compositions described herein may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles.

When aqueous suspensions and/or elixirs are desired for oral administration, the compounds described herein can be combined with various sweetening agents, flavoring agents, coloring agents, emulsifying agents and/or suspending agents, as well as such diluents as water, ethanol, propylene glycol, glycerin and various like combinations thereof. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

The combinations described herein can also be administered in a controlled release formulation (definition) such as a slow release or a fast release formulation. Such controlled release formulations of the combinations described herein may be prepared using methods well known to those skilled in the art. The method of administration will be determined, by the attendant physician or other person skilled in the art after an evaluation of the patient's condition and requirements.

Kit

The present invention also relates to a kit comprising a dosage unit of mixture of a first therapeutic agent, which is a MPO inhibitor or a pharmaceutically acceptable salt thereof and a dosage unit of a second therapeutic agent, which is a compound or a pharmaceutically acceptable salt thereof, which is used in the treatment and/or prevention of PD or MS.

One embodiment of the present invention relates to a kit as described above wherein the first therapeutic agent (a) comprises the group of compounds, which acts as MPO inhibitors as defined above.

In one embodiment of the present invention the second therapeutic agent comprises of a compound or a pharmaceutically acceptable salt thereof, which are used for the treatment and/or prevention of PD or MS as defined above. According to one embodiment of the present invention, said second therapeutic agent is selected from the group of dopamine agonists, such as those known under the generic name of bromocriptine, pergolide, ropinirole, pramipexole, cabergoline, apomorphine, pribedile and rotigotine. According to one embodiment of the present invention, said second therapeutic agent is selected from the group of MAO-B inhibitors, such as those known under the generic names of selegiline and rasagiline. According to one embodiment of the present invention, said second therapeutic agent is selected from the group of other dopaminergic compounds, such as those known under the generic names of Tolcapone, Entacapone and Budipine. According to one embodiment of the present invention, said second therapeutic agent is selected from the group of adamantane derivate, such as those known under the generic name of amantadine hydrochloride. According to one embodiment of the present invention, said second therapeutic agent is selected from the group of dopamine precursors, such as those known under the generic name of levodopa and its combination with decarboxylase inhibitors (carbidopa and benserazide) and with decarboxylase inhibitors and COMT-inhibitors (Entacapone). According to one embodiment of the present invention, said second therapeutic agent is selected from the group of Interferons, such as those known under the generic names of interferon beta-l a, interferon beta- lb, and combinations thereof. According to one embodiment of the present invention, said second therapeutic agent is selected from the group of selective immunosuppressive compounds, such as those known under the generic names of glatiramer acetate and Natalizumab.

Method of Treatment

The present invention also related to a method for treating Neuroinflammatory and Neurodegenerative Disorder(s) in a subject in need thereof comprising administering simultaneously, sequentially or separately, to said subject (a) an amount of a first therapeutic agent, which is a MPO inhibitor or a pharmaceutically acceptable salt thereof and (b) an amount of a second therapeutic agent, which is a compound or a pharmaceutically acceptable salt thereof, which is used in the treatment and/or prevention of MS or PD.

One aspect of the present invention relates to a method for treating Neuroinflammatory Disorder(s) in a subject in need thereof comprising administering simultaneously, sequentially or separately, to said subject (a) an amount of a first therapeutic agent, which is a MPO inhibitor or a pharmaceutically acceptable salt thereof and (b) an amount of a second therapeutic agent, which is a compound or a pharmaceutically acceptable salt thereof, which is used in the treatment and/or prevention of MS or PD, wherein the amounts of (a) and (b) are together synergistically effective in the treatment.

In another embodiment of the present invention, said disorder is MS or PD.

One embodiment of the present invention relates to the methods mentioned above wherein the first therapeutic agent (a) comprises of the group of compounds or pharmaceutically salts thereof that act as MPO inhibitors, which compounds have been defined above.

In one embodiment of the present invention the second therapeutic agent comprises of a compound or a pharmaceutically acceptable salt thereof, which are used for the treatment and/or prevention of PD or MS as defined above. According to one embodiment of the present invention, said second therapeutic agent is selected from the group of dopamine agonists, such as those known under the generic name of bromocriptine, pergolide, ropinirole, pramipexole, cabergoline, apomorphine, pribedile and rotigotine. According to one embodiment of the present invention, said second therapeutic agent is selected from the group of MAO-B inhibitors, such as those known under the generic names of selegiline and rasagiline. According to one embodiment of the present invention, said second therapeutic agent is selected from the group of other dopaminergic compounds, such as those known under the generic names of Tolcapone, Entacapone and Budipine. According to one embodiment of the present invention, said second therapeutic agent is selected from the group of adamantane derivate, such as those known under the generic name of amantadine hydrochloride. According to one embodiment of the present invention, said second therapeutic agent is selected from the group of dopamine precursors, such as those known under the generic name of levodopa and its combination with decarboxylase inhibitors (carbidopa and benserazide) and with decarboxylase inhibitors and COMT-inhibitors (Entacapone). According to one embodiment of the present invention, said second therapeutic agent is selected from the group of Interferons, such as those known under the generic names of interferon beta-1a, interferon beta-1b, and combinations thereof. According to one embodiment of the present invention, said second therapeutic agent is selected from the group of selective immunosuppressive compounds, such as those known under the generic names of glatiramer acetate and Natalizumab.

One aspect of the present invention relates to a method of treating Neuroinflammatory and Neurodegenerative Disorder(s), such as MS and PD, in a subject in need thereof using the kit as described above.

Another aspect of the present invention relates to a method of treating Neuroinflammatory and Neurodegenerative Disorder(s), such as MS or PD, in a subject in need thereof using the pharmaceutical composition comprising the combination comprising (a) an amount of a first therapeutic agent, which is a MPO inhibitor or a pharmaceutically acceptable salt thereof and (b) an amount of a second therapeutic agent, which is a compound or a pharmaceutically acceptable salt thereof, which is used in the treatment and/or prevention of MS or PD.

Another aspect the present invention relates to the use of the combination comprising (a) an amount of a first therapeutic agent, which is a MPO inhibitor or a pharmaceutically acceptable salt thereof and (b) an amount of a second therapeutic agent, which is a compound or a pharmaceutically acceptable salt thereof, which is used in the treatment and/or prevention of MS or PD, for the manufacturing of a medicament for use simultaneously, sequentially or separately, in therapy.

Another aspect of the present invention relates to the use of the combination comprising (a) an amount of a first therapeutic agent, which is a MPO inhibitor or a pharmaceutically acceptable salt thereof and (b) an amount of a second therapeutic agent, which is a compound or a pharmaceutically acceptable salt thereof, which is used in the treatment and/or prevention of MS or PD, for the manufacturing of a medicament for use simultaneously, sequentially or separately, in treatment of Neuroinflammatory and Neurodegenerative Disorder(s), such as MS and PD.

A further aspect of the present invention relates to an agent comprising the combination comprising (a) an amount of a first therapeutic agent, which is a MPO inhibitor or a pharmaceutically acceptable salt thereof and (b) an amount of a second therapeutic agent, which is a compound or a pharmaceutically acceptable salt thereof, which is used in the treatment and/or prevention of MS or PD, for use simultaneously, sequentially or separately, for treatment of Neuroinflammatory Disorder(s), such as MS and PD.

Dosage

The effective dose of a MPO inhibitor or a pharmaceutically acceptable salt thereof and a compound or a pharmaceutically acceptable salt thereof, which is used in the treatment and/or prevention of MS or PD in the combinations according to the present invention may vary, depending upon factors such as the condition of the patient, the severity of the symptoms of the disorder as well as the potency of the selected specific compound, the mode of administration, the age and weight of the patient, and the like. Determining a dose is within the skill of the ordinary artisan.

The exact formulation, route of administration, and dosage can be chosen by the individual physician in view of the patient's condition. Dosage amount and interval can be adjusted individually to provide plasma levels of the active moiety, which are sufficient to maintain therapeutic effects.

Typically, the effective dose of MPO inhibitors or a pharmaceutically acceptable salt thereof generally requires administering the compound in a range of from, and including, 1 to 1 000 mg. According to one embodiment of the present invention, said range is from, and including, 2 to 800 mg or from, and including, 2 to 400 mg. In an alternative embodiment of the present invention the amount of MPO inhibitor or a pharmaceutically acceptable salt thereof is selected from about: 5, 10, 50, 100, 150, 200, 250, 300, 350, 400, 500, 550, 600, 700 and 800 mg.

Typically, the effective dose compound or a pharmaceutically acceptable salt thereof, which is used in the treatment and/or prevention of MS or PD generally requires administering the compound in a range of from, and including, 1 to 1 000 mg. According to one embodiment of the present invention, said range is from, and including, 2 to 800 mg or from, and including, 2 to 400 mg. In an alternative embodiment of the present invention the amount of compound or a pharmaceutically acceptable salt thereof, which is used in the treatment and/or prevention of MS or PD is selected from about: 5, 10, 50, 100, 150, 200, 250, 300, 350, 400, 500, 550, 600, 700 and 800 mg.

The term “therapeutically-effective amount” as used herein refers to a sufficient amount of the compound to treat neuroinflammatory and neurodegenerative disorders at a reasonable risk/benefit ratio applicable to any medical treatment.

The term “treating” as used herein, refers to reversing, alleviating, delaying or inhibiting the progress of, or preventing the disorder or condition to which such term applies, or one or more symptoms of such disorder or condition. The term “treatment”, as used herein, refers to the act of “treating” as defined herein.

The term “disorder”, unless stated otherwise, has the same meaning as the terms “condition” and “disease” and are used interchangeably throughout the description and claims.

The term “agent” means the compounds comprised in the combination of the present invention. The exact formulation, route of administration, and dosage can be chosen by the individual physician in view of the patient's condition. Dosage amount and interval can be adjusted individually to provide plasma levels of the active moiety, which are sufficient to maintain therapeutic effects.

According to the present invention the term neurological diseases is intended to include both Neuroinflammatory and Neurodegenerative Disorder(s) In the present invention, the term “Neuroinflammatory and Neurodegenerative Disorder(s)” is intended to include Multiple Sclerosis (MS), Parkinson's disease, Multiple System Atrophy (MSA), Corticobasal Degeneration, Progressive Supranuclear Paresis, Guillain-Barré Syndrome (GBS), chronic inflammatory demyelinating polyneuropathy (CIDP),

Also, the term Neurodegenerative Disorder(s) is intended o include

a) Dementia, including but not limited to Alzheimer's Disease (AD), Down syndrome, vascular dementia, Parkinson's Disease (PD), HIV dementia, Huntington's Disease, amyotrophic lateral sclerosis (ALS), motor neuron diseases (MND), progressive supranuclear palsy (PSP), Pick's Disease, Niemann-Pick's Disease, corticobasal degeneration, traumatic brain injury (TBI), dementia pugilistica, Creutzfeld-Jacob Disease and prion diseases;

b) Cognitive Deficit in Schizophrenia (CDS);

c) Mild Cognitive Impairment (MCI);

d) Age-Associated Memory Impairment (AAMI);

e) Age-Related Cognitive Decline (ARCD);

f) Cognitive Impairement No Dementia (CIND).

In the present invention, the term “Multiple Sclerosis (MS) is intended to also include Relapse Remitting Multiple Sclerosis (RRMS), Secondary Progressive Multiple Sclerosis (SPMS), and Primary Progressive Multiple Sclerosis (PPMS).

According to the present invention the term “Parkinson's Disease (PD)” is intended to also include postencephelatic parkinsonism, dementia with Lewy bodies, and Frontotemporal dementia Parkinson's Type (FTDP).

1. A combination comprising (a) an amount of a first therapeutic agent, which is a MPO inhibitor or a pharmaceutically acceptable salt thereof and (b) an amount of a second therapeutic agent, which is a compound or a pharmaceutically acceptable salt thereof, which is used in the treatment and/or prevention of Parkinson's Disease or Multiple Sclerosis.

2. A pharmaceutical composition comprising a combination comprising (a) an amount of a first therapeutic agent, which is MPO inhibitor or a pharmaceutically acceptable salt thereof and (b) an amount of a second therapeutic agent, which is a compound or a pharmaceutically acceptable salt thereof, which is used in the treatment and/or prevention of Parkinson's Disease or Multiple Sclerosis, together with a pharmaceutically-acceptable vehicle, carrier and/or diluent.

3. The pharmaceutical composition according to claim 2, wherein the second therapeutic agent is selected from dopamine agonists MAO-B inhibitors, and other dopaminergic compounds, adamantane derivates dopamine precursors, decarboxylase inhibitors, COMT-inhibitors, and combinations thereof.

4. The pharmaceutical composition according to claim 2, wherein the second therapeutic agent is selected from Interferons and selective immunosuppressive compounds.

5. A kit comprising a dosage unit of mixture of a first therapeutic agent, which is a MPO inhibitor or a pharmaceutically acceptable salt thereof and a dosage unit of a second therapeutic agent, which is a compound or a pharmaceutically acceptable salt thereof, which is used in the treatment and/or prevention of Parkinson's Disease or Multiple Sclerosis, optionally with instructions for use.

6. A method for treating Neuroinflammatory Disorder(s) in a subject in need thereof comprising administering simultaneously, sequentially or separately, to said subject (a) an amount of first therapeutic agent, which is a MPO inhibitor or a pharmaceutically acceptable salt thereof and (b) an amount of a second therapeutic agent, which is a compound or a pharmaceutically acceptable salt thereof, which is used in the treatment and/or prevention of Parkinson's Disease or Multiple Sclerosis, wherein the amounts of (a) and (b) are together synergistically effective in the treatment.

7. The method according to claim 6, wherein said disorder is Multiple Sclerosis or Parkinson's Disease.

8. The method according to claim 6 or claim 7, wherein the second therapeutic agent is selected from dopamine agonists, MAO-B inhibitors, other dopaminergic compounds, adamantane derivate, dopamine precursors, decarboxylase inhibitors, COMT-inhibitors, and mixtures thereof.

9. The method according to claim 6 or claim 7, wherein the second therapeutic agent is selected from Interferons and selective immunosuppressive compounds.

10. A method for treating Neuroinflammatory Disorder(s) in a subject in need thereof with a kit in accordance with claim 5, wherein said first therapeutic agent and said second therapeutic agent are administered simultaneously sequentially or separately to said subiect and said first therapeutic agent and said second therapeutic agent are administered in amounts that are together synergistically effective in treating said Neuroinflammatory Disorder(s).

11. A method for treating Neuroinflammatory Disorder(s) in a subiect in need thereof comprising administering an effective amount of a pharmaceutical composition according to claim 2, wherein said pharmaceutical composition is administered orally, transmucosally, transdermally, nasaly, pulmonarily, buccally, parenterally, rectally, sublingually, intravenously, intramuscularly, subcutaneously, or intradermally.

12. The method according to claim 11, wherein said pharmaceutical composition is administered intravenously, intramuscularly, subcutaneously, or intradermally.

13. The method according to claim 11, wherein said pharmaceutical composition is administered orally, transmucosally, transdermally, nasally, pulmonarily, buccally, parenterally, rectally, or sublingually.
